(19) United States
(12) Patent Application Publication
(10) Pub. No.: US 2007/0225798 A1
(43) Pub. Date: Sep. 27, 2007

(54) Side branch stent

(76) Inventor: Daniel Gregorich, St. Louis Park, MN (US)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 11/387,560

(22) Filed: Mar. 23, 2006

(51) Int. Cl. A61F 2/06

(52) U.S. Cl. 623/1.35

(57) ABSTRACT

The present invention is directed to a medical device for expansion in a bifurcated bodily vessel. The medical device includes a first stent and at least one expansion column connected to an end of the first stent. The axes of the first stent and the expansion column(s) are generally perpendicularly oriented relative to one another, such that the expansion column(s) is/are expanded within the main branch of the bifurcated bodily vessel and the first stent is expanded within a side branch of the bifurcated bodily vessel.

### BACKGROUND OF THE INVENTION

1. Field of the Invention

In some embodiments this invention relates to implantable medical devices, their manufacture, and methods of use. Some embodiments are directed to tubular stents having perpendicularly oriented expansion columns extending from the ends of the tubular stents and delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature, it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel) two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In some embodiments this invention relates to tubular stents having perpendicularly oriented expansion columns extending from the ends of the tubular stents and delivery systems, such as catheter systems of all types, which are utilized in the delivery of such devices, for treating bifurcated vessels.

In at least one embodiment, the invention is directed to a medical device having a stent and a first expansion column connected to and extending from the proximal end of the stent. The axis of the expansion column is at an angle relative to the axis of the stent.

In at least one embodiment, the invention is directed to a medical device having two expansion columns connected to and extending from the proximal end of the stent. The axes of the expansion columns are at an angle relative to the axis of the stent. The axis of the first expansion column and the axis of the second expansion column may also be substantially aligned.

In at least one embodiment, the invention is directed to a medical device having a stent and an expansion column connected to and extending from the proximal end of the stent. The axis of the expansion column is at an angle relative to the axis of the stent and the angle relative to the axis of the stent may be flexibly adjustable.

In at least one embodiment, the invention is directed to a medical device having a stent and one or more expansion columns connected to and extending from the proximal end of the stent. The axis of the expansion column(s) is at an angle relative to the axis of the stent and the expansion column(s) is formed from a petal-shaped configuration.

In at least one embodiment, the invention is directed to a medical device having a stent and one or more expansion columns connected to and extending from the proximal end of the stent. The axis of the expansion column(s) is at an angle relative to the axis of the stent. The stent may be formed from a plurality of expansion columns, each expansion column of the stent being connected to a longitudinally adjacent expansion column of the stent. The axis of the stent may be substantially perpendicularly oriented relative to the axis of the expansion column(s).

In at least one embodiment, the invention is directed to a medical device having a stent and one or more expansion columns connected to and extending from the proximal end of the stent, wherein the expansion column(s) is connected to the stent by no more than one connection. The axis of the expansion column(s) is at an angle relative to the axis of the stent and the expansion column(s) is formed from a petal-shaped configuration.

In at least one embodiment, the invention is directed to an assembly including a delivery catheter and a mounted medical device having a stent and one or more expansion columns connected to and extending from the proximal end of the stent. The axis of the expansion column is at an angle relative to the axis of the stent.

In at least one embodiment, the invention is directed to a method of stenting a vessel. The method includes providing an assembly including a delivery catheter and a mounted medical device having a stent and one or more expansion columns connected to and extending from the proximal end of the stent. The axis of the expansion column is at an angle relative to the axis of the stent. The method further includes the step of delivering the medical device to a deployment location in a bodily vessel and then expanding the medical device. The deployment location may be a bifurcation in the vessel.

In at least one embodiment, the invention is directed to a medical device comprising a stent, wherein the stent is formed by rolling a flat pattern into a tubular shape. The flat pattern has a first end and a second end, wherein the first end is opposite the second end. One or more petal-shaped extensions extend from the first end of the stent flat pattern.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described an embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a top view of a flat pattern for an embodiment of a medical device.

FIG. 1A is a top view of a flat pattern of an isolated expansion column for an embodiment of a medical device.

FIG. 2 is a top view of a petal for an embodiment of a medical device.

FIG. 3 is a side view of an embodiment of a medical device.

FIG. 4 is a side view of an embodiment of a medical device.

FIG. 4A is a side view of expansion column of an embodiment of a medical device.

FIG. 5 is an end view of an embodiment of a medical device.

FIG. 6 is a side view of an embodiment of a medical device.

FIG. 7 is a side view of an embodiment of a medical device.

FIG. 8 is a side view of an embodiment of an expanded medical device positioned within a bifurcated bodily vessel.

FIG. 9 is a side view of an embodiment of a medical device mounted on a balloon.

FIG. 10 is a side view of an embodiment of a medical device mounted on a balloon.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

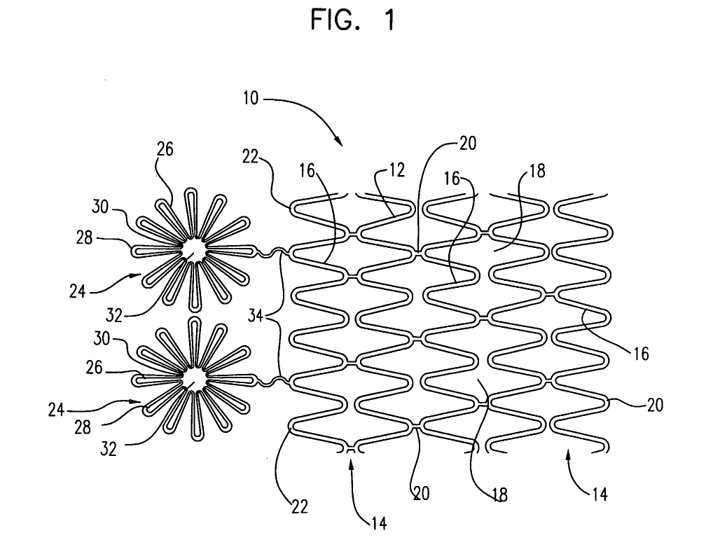

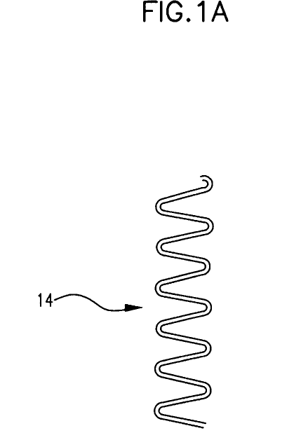

Referring now to the drawings, which are for the purposes of illustrating embodiments of the invention only and not for purposes of limiting same, FIG. 1 illustrates an embodiment of the side branch stent in its unrolled or flat pattern after it has been cut or etched from a flat sheet of material. A person of ordinary skill in the art will recognize that the flat pattern may be rolled to form a cylindrical medical device. It should be understood that stents may be created by many known methods, including, but not limited to, cutting or etching a design from a tubular stock or from a flat sheet of suitable material and subsequently rolled or from one or more interwoven wires or braids.

In its flat sheet form, as shown in FIG. 1, the side branch stent, generally designated as 10, includes a first stent 12. The first stent 12 may take the form and design of any expandable stent, including self-expanding stents. In this particular embodiment, the first stent 12 includes expansion columns 14. An isolated expansion column 14 is shown in FIG. 1A. Five vertically oriented expansion columns 14 are shown in FIG. 1. However, it should be understood that the number of expansion columns may vary according to intended use.

The first stent 12 comprises a plurality of interconnected struts 16 defining a plurality of cells 18. In this particular embodiment, the interconnected struts 16 of the individual expansion columns 14 form a serpentine pattern, which is shown to be vertically oriented in FIG. 1. The adjacent expansion columns 14 may be directly connected to each other or they be connected via connecters 20. The amount and orientation of the connection points may vary. While “serpentine” may be used to describe most embodiments, the term is not intended to limit the invention. It should be understood that the present invention is not limited to any particular stent pattern for the first stent 12. One skilled in the art would recognize that other suitable patterns may be used.

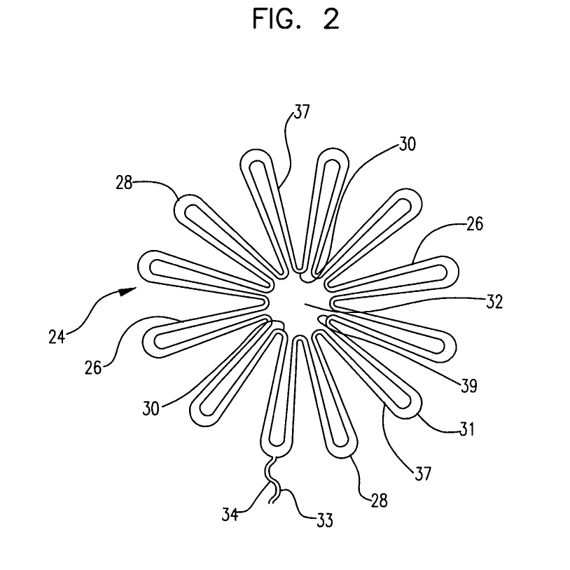

Connected to the proximal end 22 of the first stent 12 are one or more petals 24. A blown up illustration of a petal 24 is shown in FIG. 2. The configuration of the petal 24 shown in FIG. 2 is considered to be a flattened configuration.

Two petals 24 are shown in the embodiment of FIG. 1. In the flat pattern, as shown in FIG. 1, each petal 24 may comprise a plurality of struts 26, which are separated by outer bends 28, the apex 31 of which points away from the center 32, and inner 30 bends, the apex 39 of which points toward the center 32. Each pairing of adjacent struts 26 is considered to be a peak 37. FIG. 2 shows 12 peaks around the petal 24, however, the present invention also contemplates embodiments having 3-12 peaks. A strut 26 may be straight or slightly curved along its length and may be oriented in any suitable direction. A bend 28, 30 may be oriented in any suitable direction and, in some embodiments, may be oriented toward the center 32 of the petal 24.

The petals 24 are connected directly to the proximal end 22 of the first stent 12 or via connecters 34. The petals 24 may be separate pieces which are connected to the first stent 12 or they may be integrally formed with the first stent 12 from a single piece of material via etching, cutting or by other suitable methods. The connectors 34 may be straight pieces or they may incorporate one or more undulating bends 33 to afford bending flexibility.

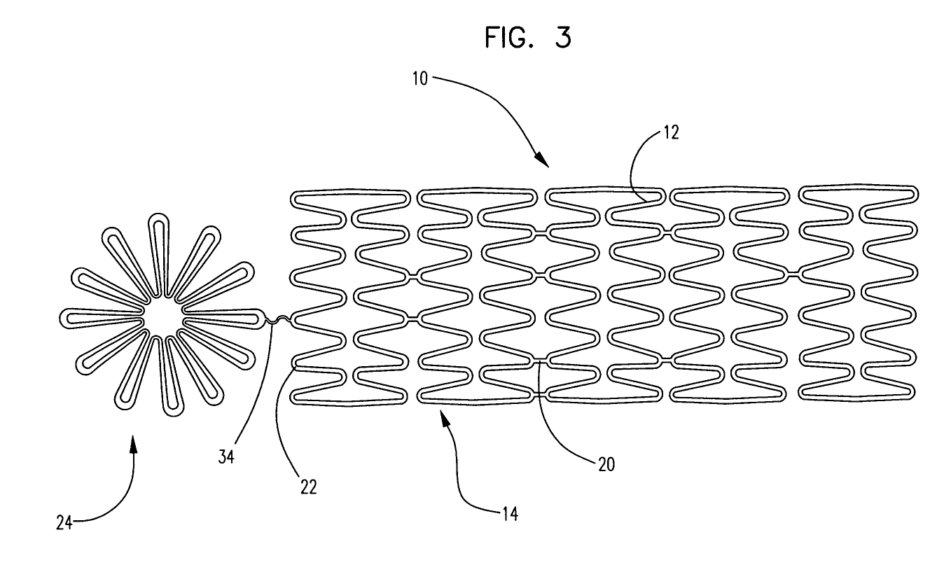

FIG. 3 illustrates a side view of the side branch stent 10 after the first stent 12 flat pattern has been rolled to form a cylindrical medical device. Only one petal 24 is shown connected to the proximal end 22 of the first stent 12, as the second petal is hidden behind the first petal. The design of the first stent 12 allows for the contraction and expansion of the diameter of the first stent 12.

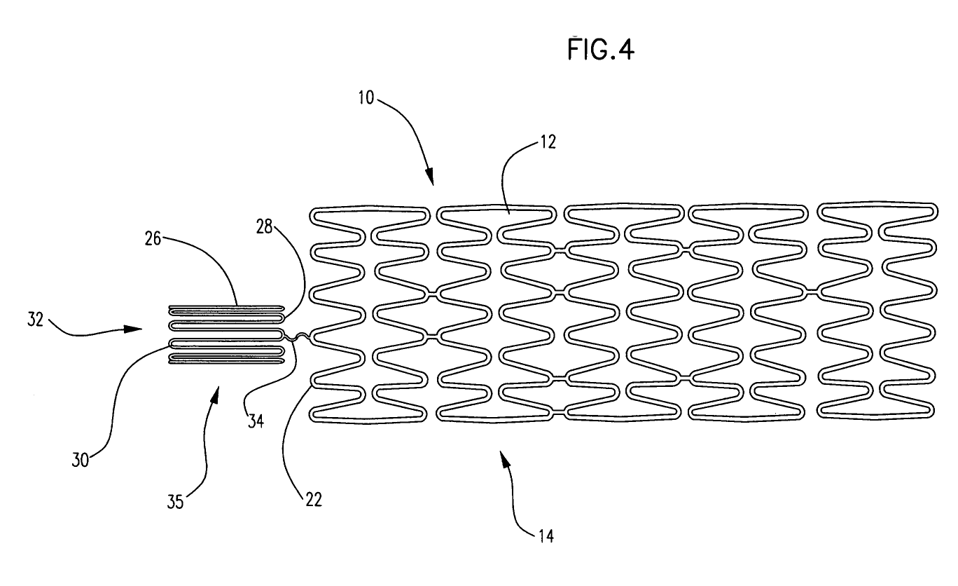

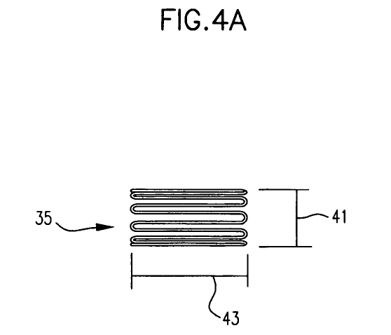

The conversion of the petal 24 shown in FIG. 3 to a contracted cylindrical form 35 is illustrated in FIG. 4. The contracted form 35, hereafter referred to as a contracted petal 35, in the embodiments shown is considered to be an expansion column which may be radially expandable, as shown in later figures. As shown in FIG. 4A, the contracted petal 35, or expansion column, has a width 43 and a diameter 41. This is as apposed to the petal 24 shape, which is planer or flattened. As the expansion column 35 expands from a contracted state to an expanded state, the diameter 41 increases and the width 43 decreases.

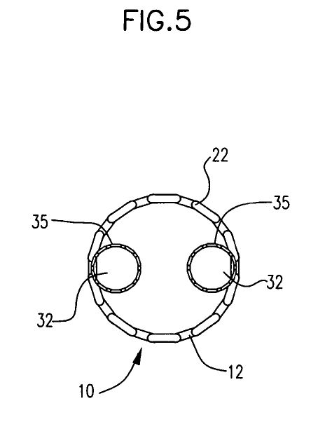

In FIG. 4, only one contracted petal 35 is shown is shown in the illustration. The remaining petal 24 is blocked from view. Referring to FIG. 2, the conversion of the petals 24 may be achieved by drawing the outer bends 28 toward one another while maintaining the positions of the inner bends 30 relative to one another. In doing so, the outer bends 28 are drawn closer to connector 34. FIG. 5 is a representation view of the proximal end of the side branch stent 10 after this conversion. Two contracted petals 35 are shown extending from the proximal end 22 of the first stent 12. Alternatively, the petals can be rotated outward about the proximal end of the stent before crimping, forming a “T-shaped” stent.

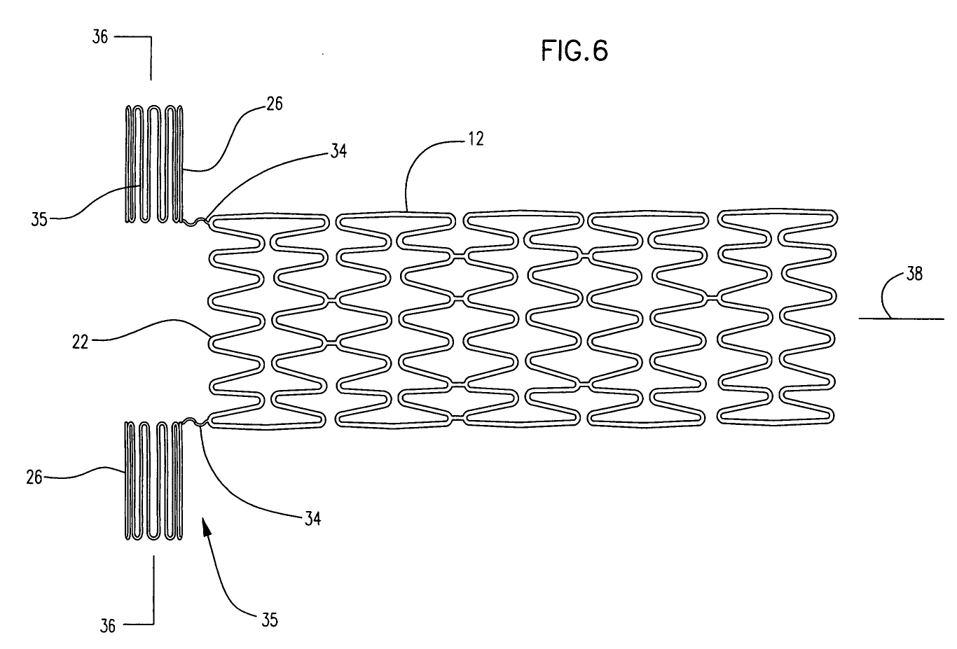

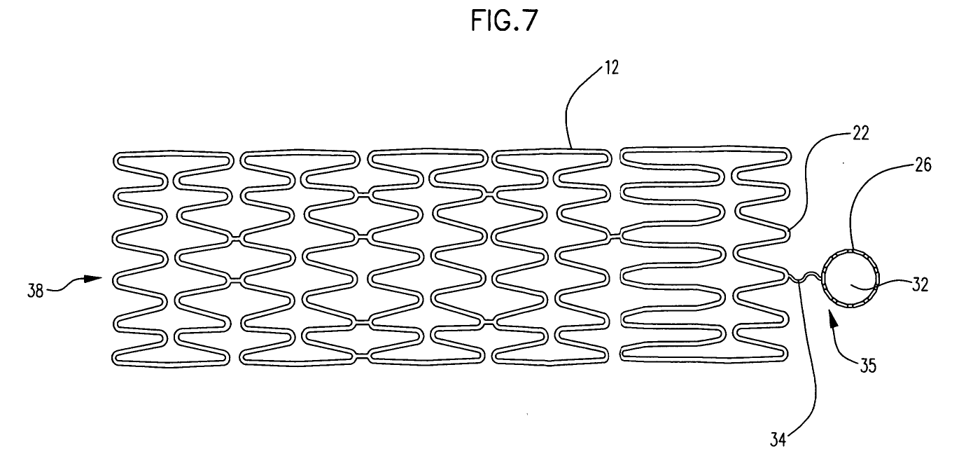

In FIGS. 6 and 7, the contracted petals 35 are rotated outward about the proximal end of the first stent 12. The flexibility of the connectors 34 allow for this rotation, such that the axes 36 of the contracted petals 35 may be adjusted relative to the axis 38 of the first stent 12. When in place, the axis 36 of each of the contracted petals 35 forms an angle of at least 90° with the axis 38 of the first stent 12. FIG. 6 illustrates a view in which the sides of the contracted petals 35 may be seen and FIG. 7 illustrates a view in which the end of the contracted petals may be seen. In the view shown in FIG. 7, one contracted petal 35 is blocked from view by the one shown.

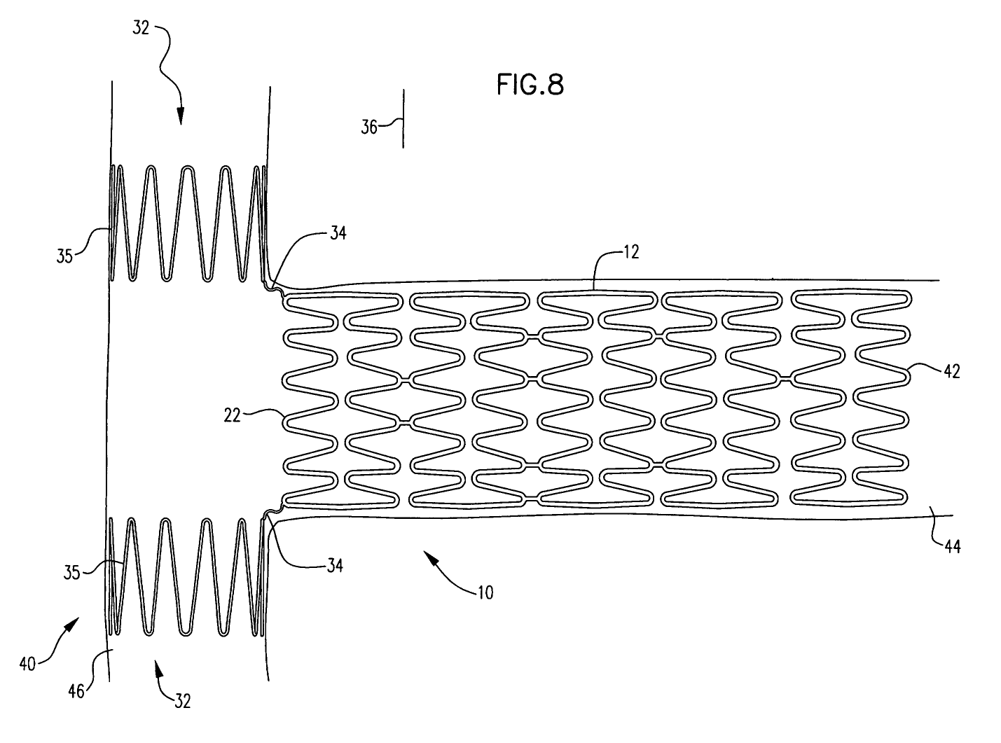

The side branch stent 10 may be crimped on and delivered by either a regular or specialized delivery balloon and advanced until the first stent 12 is in a side branch of a bodily vessel with the contracted petal(s) 35 hanging out in the main branch of the vessel. As shown in FIG. 8, the side branch stent 10 is positioned in a bifurcated vessel location 40. In the delivery of the side branch stent 10, the leading distal end 42 is first positioned in the side branch 44 leaving the contracted petals 35 (shown as expanded) in the main branch 46 of the vessel location 40. The final expanded positioning is illustrated in FIG. 8. The axis of first stent 12 is substantially aligned with the axis of the side branch 44 and the axes of the contracted petals 35 are substantially align with each other and the axis of the main branch 46.

The contracted petals 35 may be expanded individually or simultaneously, before or after the expansion of the first stent 12 via balloon or self expansion. In one embodiment, the inflation of the side branch balloon is followed by the expansion of the main branch balloon. A specialized T-shaped balloon may also be used to expand the contracted petal(s) 35 and the first stent 12 at the same time. Multiple balloons which form a “T” shape are also contemplated in the expansion of the side branch stent 10. The multiple balloons may have the same or separate inflation lumens. The catheter used may have guidewire ports to facilitate the use of ancillary devices or post-dilatation balloons for the main branch and/or the side branch, as well as have the ability to keep the guidewire in the vasculature after removal of the device.

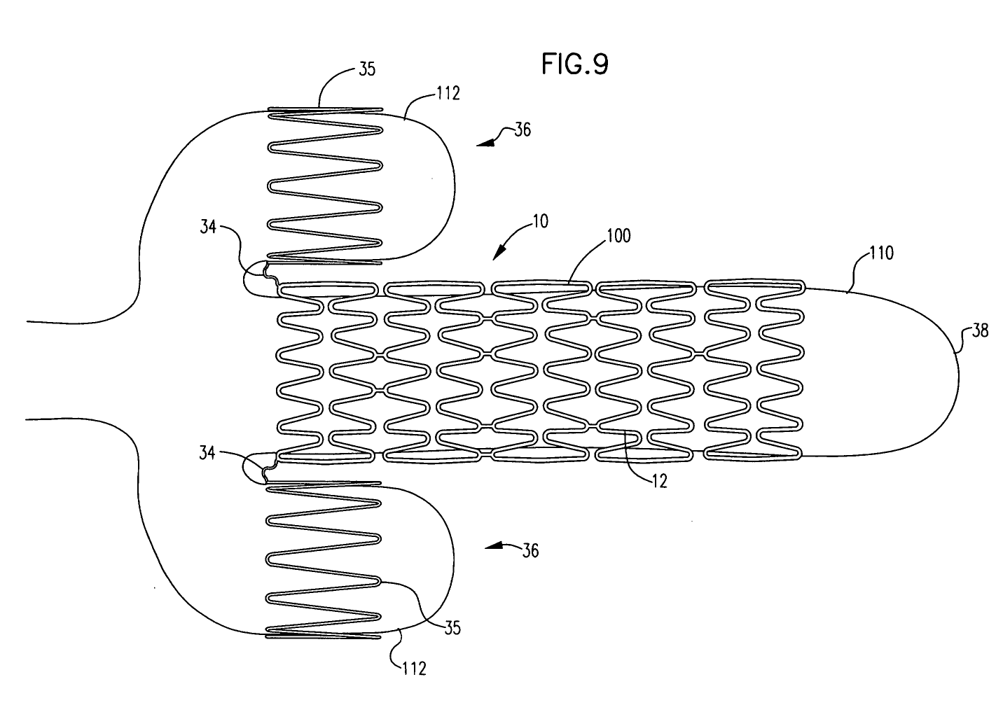

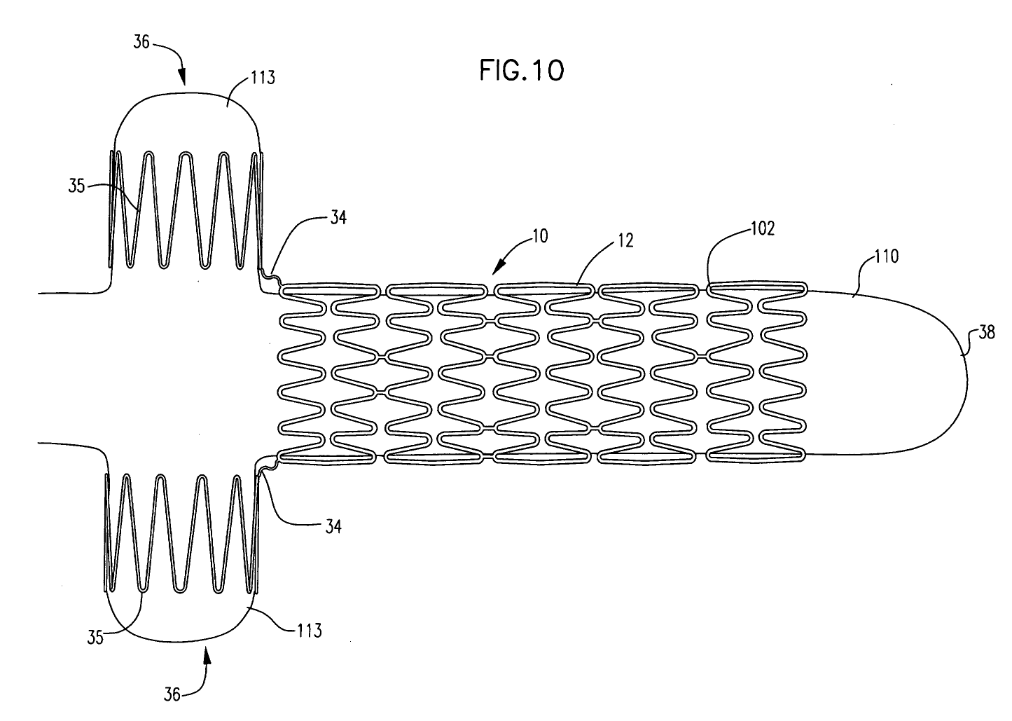

FIGS. 9-10 illustrates the side branch stent 10 mounted and expanded on two variations of a T-shaped balloon. It should be understood that other balloon types besides the ones shown are contemplated in the present invention. In FIG. 9, the balloon 100 has a main body 110 and two branches 112. For the balloon 100 shown, the branches 112 extend radially out from the main body 110 and then turn parallel to the main body 110. The contracted petals 35 are about the branches 110 and the first stent 12 is about the main body 110. In this configuration, the axes 36 of the contracted petals 35 are largely parallel with the axis 38 of the first stent 12. The embodiment shown in FIG. 10 differs from the embodiment shown in FIG. 9 in that the side branches 113 of the balloon 102 extend directly radially from the main body 110 and the contracted petals 35 are mounted such that the axes 36 of the contracted petals 35 are largely perpendicular to the axis 38 of the first stent 12.

The side branch stent 10 of the present invention may be used with bifurcated catheter systems. Examples of bifurcated systems include, but are not limited to, those shown and described in U.S. patent application Ser. No. 10/375,689, filed Feb. 27, 2003 and U.S. patent application Ser. No. 10/657,472, filed Sep. 8, 2003, both of which are entitled Rotating Balloon Expandable Sheath Bifurcation Delivery; U.S. patent application Ser. No. 10/747,546, filed Dec. 29, 2003 and entitled Rotating Balloon Expandable Sheath Bifurcation Delivery System; U.S. Published Application No. 20050060027; and U.S. patent application Ser. No. 10/757,646, filed Jan. 13, 2004, and entitled Bifurcated Stent Delivery System, the entire content of each being incorporated herein by reference.

The contracted petals 35 geometry and/or the first stent 12 geometry may be self-expanding, balloon expandable or a combination thereof. It should also be understood that the contracted petals 35 may be a separate entity from the first stent 12 in that they have different geometric designs.

The inventive stents may be made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, magnesium, tungsten, gold and alloys of any of the above-mentioned metals. Examples of suitable alloys include platinum-iridium alloys, magnesium based alloys, cobalt-chromium alloys including Elgiloy and Phynox, MP35N alloy and nickel-titanium alloys, for example, Nitinol.

The inventive stents may be made of shape memory materials such as superelastic Nitinol or spring steel, or may be made of materials which are plastically deformable. In the case of shape memory materials, the stent may be provided with a memorized shape and then deformed to a reduced diameter shape. The stent may restore itself to its memorized shape upon being heated to a transition temperature and having any restraints removed therefrom.

The inventive stents may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments the at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto.

1. A medical device comprising:
a first stent, the first stent comprising a distal end, a proximal end, and an opening extending from the distal end to the proximal end along an axis, wherein the first stent is expandable from a contracted state to an expanded state; and
a first expansion column, the first expansion column comprising a first end, a second end, a width defined by the distance between the first end and the second end and an opening having a diameter, the opening extending from the first end to the second end along an axis, wherein the first expansion column is expandable from a contracted state to an expanded state,
wherein the first expansion column is connected to the proximal end of the first stent and the axis of the at least one expansion column is at an angle relative to the axis of the first stent and wherein the first expansion column is deformable from and/or into a flattened configuration, such that, when in the flattened configuration, portions that make up the first end of the first expansion column are surrounded by portions that make up the second end of the first expansion column.

2. The medical device of claim 1, further comprising a second expansion column, the second expansion column comprising a first end, a second end, and an opening extending from the first end to the second end along an axis, wherein the second expansion column is expandable from a contracted state to an expanded state and wherein the second expansion column is connected to the proximal end of the first stent and the axis of the second expansion columns is at an angle relative to the axis of the first stent and wherein the second expansion column is deformable from and/or into a flattened configuration, such that, when in the flattened configuration, portions that make up the first end of the second expansion column are surrounded by portions that make up the second end of the second expansion column.

3. The medical device of claim 2, wherein the axis of the first expansion column and the axis of the second expansion column are substantially aligned.

4. The medical device of claim 1, wherein the angle relative to the axis of the first stent is flexibly adjustable.

5. The medical device of claim 1, wherein the first expansion column is formed from a petal-shaped configuration.

6. The medical device of claim 2, wherein the first and second expansion columns are formed from petal-shaped configurations.

7. The medical device of claim 1, wherein the first stent comprises a plurality of expansion columns, each expansion column of the first stent being connected to a longitudinally adjacent expansion column of the first stent, wherein the proximal end of the first stent is defined by a proximal expansion column, the proximal expansion column being the most proximally positioned expansion column of the first stent, wherein the first expansion column is connected to the proximal expansion column.

8. The medical device of claim 2, wherein the first stent comprises a plurality of expansion columns, each expansion column of the first stent being connected to a longitudinally adjacent expansion column of the first stent, wherein the proximal end of the first stent is defined by a proximal expansion column, the proximal expansion column being the most proximally positioned expansion column of the first stent, wherein the first and second expansion columns are connected to the proximal expansion column.

9. The medical device of claim 8, wherein the axis of the first stent is substantially perpendicularly oriented relative to the axes of the first and second expansion columns.

10. The medical device of claim 1, wherein the expansion columns of the first stent comprises a first strut pattern, the first strut pattern comprising a plurality of alternating peaks, wherein a plurality of peaks of each expansion column of the first stent are connected to an adjacent expansion column of the first stent.

11. The medical device of claim 1, wherein the first expansion column comprises a first strut pattern, the first strut pattern comprising a plurality of alternating peaks, wherein at least one peak of the first strut pattern of the first expansion column is connected to the proximal end of the first stent.

12. The medical device of claim 2, wherein the first and second expansion columns each comprise a first strut pattern, the first strut patterns each comprising a plurality of alternating peaks, wherein at least one peak of the first strut patterns of the first and second expansion columns are connected to the proximal end of the first stent.

13. The medical device of claim 1, wherein the first stent is connected to the first expansion column by no more than one connection.

14. The medical device of claim 1, wherein the first stent is connected to each of the first and second expansion columns by no more than one connection.

15. An assembly comprising:
a delivery catheter comprising a catheter shaft having an inner lumen; and
the medical device of claim 1, wherein the medical device is oriented about a distal portion of the catheter shaft.

16. The assembly of claim 15, the medical device of claim 1 further comprising a second expansion column, the second expansion column comprising a first end, a second end, and an opening extending from the first end to the second end along an axis, wherein the second expansion column is expandable from a contracted state to an expanded state and wherein the second expansion column is connected to the proximal end of the first stent and the axis of the second expansion columns is at an angle relative to the axis of the first stent and wherein the second expansion column is deformable from and/or into a flattened configuration, such that, when in the flattened configuration, portions that make up the first end of the second expansion column are surrounded by portions that make up the second end of the second expansion column.

17. The assembly of claim 16, wherein the delivery catheter further comprises a first inflation balloon, at least a portion of the medical device oriented about the first inflation balloon.

18. The assembly of claim 17, wherein the delivery catheter further comprises a second inflation balloon, wherein the first stent is oriented about the first inflation balloon and the first and/or second expansion column is/are oriented about the second inflation balloon.

19. A method of stenting a vessel comprising:
providing the assembly of claim 16;
delivering the medical device to a deployment location in a bodily vessel; and
expanding the medical device.

20. The method of claim 19, the bodily vessel at the deployment location having a main branch and a side branch extending from the main branch, wherein the first stent is expanded in the side branch and the first and second expansion columns are expanded in the main branch.

21. A medical device, the medical device comprising a stent, wherein the stent is formed by rolling a flat pattern into a tubular shape, the flat pattern having a first end and a second end, wherein the first end is opposite the second end, the medical device having at least one petal-shaped extension extending from the first end of the flat pattern of the stent.

22. The medical device of claim 21, wherein the stent has two petal-shaped extensions extending from the first end of the flat pattern of the stent.
